(19) United States
(12) Patent Application Publication
(10) Pub. No.: US 2007/0197769 A1
(43) Pub. Date: Aug. 23, 2007

(54) Telomerization processes

(76) Inventors: Stephan Brandstadter, Indianapolis, IN (US); Bruno Ameduri, Montpelier (FR); George K. Kostov, Montpelier (FR)

Correspondence Address: WELLS ST. JOHN P.S., 601 W. FIRST AVENUE, SUITE 1300, SPOKANE, WA, 99201, US

(21) Appl. No.: 11/784,446

(22) Filed: Apr. 5, 2007

Related U.S. Application Data
(62) Division of application No. 11/192,832, filed on Jul. 28, 2005.

(51) Int. Cl. C07C 17/20

(52) U.S. Cl. 528/397; 528/401; 570/170

(57) ABSTRACT

Compositions are provided that can include R_{F}(R_{T})ₙQ, 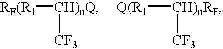
and/or R_{Cl}(R_{T})ₙH. The R_{F} group can have four fluorine atoms, the R_{T} group can include a C-2 group having a pendant —CF₃ group, n can be at least 1, the R₁ group can include a carbon atom, the R_{Cl} group can be —CCl₃, and the Q group can include one or more atoms of the periodic table of elements. Telomerization processes are also provided.

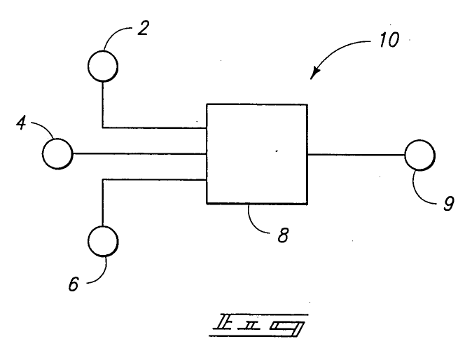

### TECHNICAL FIELD

The disclosure pertains to compositions, halogenated compositions, chemical production and telomerization processes.

### BACKGROUND

Compositions such as surfactants, polymers, and urethanes have incorporated halogenated functional groups. These functional groups have been incorporated to affect the performance of the composition when the composition is used as a treatment for materials and when the composition is used to enhance the performance of materials. For example, surfactants incorporating halogenated functional groups can be used as fire extinguishants either alone or in formulations such as aqueous film forming foams (AFFF). Polymers and/or urethanes incorporating halogenated functional groups have also been used to treat materials. To prepare these compositions, halogenated intermediate compositions can be synthesized.

### SUMMARY

Compositions are provided that can include R_{F}(R_{T})ₙQ and/or one or both of 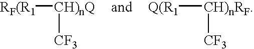
Within these compositions the R_{F} group can have at least four fluorine atoms, the R_{T} group can include at least one C-2 group having at least one pendant —CF₃ group, n can be at least 1, the R₁ group can include at least one carbon atom, and the Q group can include one or more atoms of the periodic table of elements. Compositions are provided that can also include R_{Cl}(R_{T})ₙH, with the R_{Cl} group having at least one —CCl₃ group.

Telomerization processes are also provided that include exposing at least one CF₃-comprising taxogen to a fluorine-comprising telogen to produce a telomer, with the fluorine-comprising telogen including at least four fluorine atoms.

### BRIEF DESCRIPTION OF THE DRAWING

The FIGURE is a diagram of a system according to an exemplary embodiment of an exemplary aspect of the invention.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

Compositions and methods of making compositions are described with reference to the FIGURE. Referring to the FIGURE, a system 10 is shown for preparing halogenated compositions that includes reagents such as a taxogen 2, a telogen 4, and an initiator 6 being provided to reactor 8 to form a product such as a telomer 9. In exemplary embodiments system 10 can perform a telomerization process. According to an embodiment, taxogen 2 can be exposed to telogen 4 to form telomer 9. In accordance with another embodiment, taxogen 2 can be exposed to telogen 4 in the presence of initiator 6. Reactor 8 can also be configured to provide heat to the reagents during the exposing.

Taxogen 2 can include at least one CF₃-comprising compound. The CF₃-comprising compound can have a C-2 group having at least one pendant —CF₃ group. In exemplary embodiments taxogen 2 can comprise an olefin, such as 3,3,3-trifluoropropene (TFP, trifluoropropene) and/or 1,1,3,3,3-pentafluoropropene (PFP, pentafluoropropene).

Telogen 4 can include halogens such as fluorine and/or chlorine. Telogen 4 can include at least four fluorine atoms and can be represented as R_{F}Q and/or R_{Cl}Q. The R_{F} group can include at least four fluorine atoms and the Q group can include one or more atoms of the periodic table of elements. The Q group can be H or I with the R_{F} group being (CF₃)₂CF— and/or —C₆F₁₃, for example. The R_{Cl} group can include at least one —CCl₃ group. Exemplary telogens can include (CF₃)₂CFl, C₆F₁₃I, trichloromethane, HP(O)(OEt)₂, BrCFClCF₂Br, R-SH (R being a group having carbon), and/or MeOH. In exemplary embodiments, taxogen 2 can include trifluoropropene and telogen 4 can include (CF₃)₂CFl, with a mole ratio of taxogen 2 to telogen 4 being from about 1:1 to about 1:10, 1:4 to about 4:1, and/or to about 2:1 to about 4:1.

Reactor 8 can be any lab-scale or industrial-scale reactor and, in certain embodiments, reactor 8 can be configured to control the temperature of the reagents therein. According to exemplary embodiments reactor 8 can be used to provide a temperature during the exposing of the reagents of from about 130° C. to about 150° C.

Telomer 9, produced upon exposing taxogen 2 to telogen 4, can include R_{F}(R_{T})ₙQ and/or R_{Cl}(R_{T})ₙH. The R_{T} group can include at least one C-2 group having a pendant —CF₃ group, such as 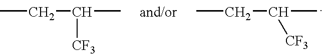
Exemplary products include 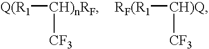
and/or one or both of 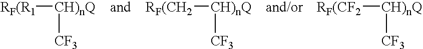
with R₁ including at least one carbon atom, such as —CH₂— and/or —CF₂—, for example. In exemplary embodiments, n can be at least 1 and in other embodiments n can be at least 2 and the product can include one or more of

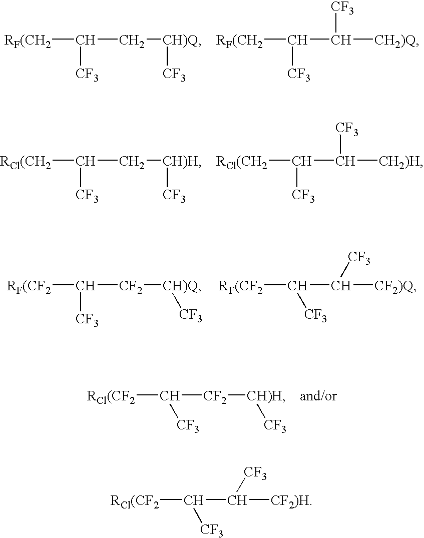

In an exemplary embodiment, the taxogen trifluoropropene can be exposed to the telogen (CF₃)₂CFl to form the telomer 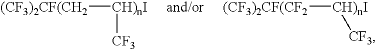
and, by way of another example, trifluoropropene can be exposed to the telogen C₆F₁₃I to form the telomer 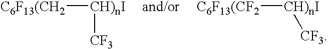
In accordance with another embodiment, the taxogen trifluoropropene can also be exposed to the telogen CCl₃Z, (Z=H, Br and/or Cl, for example) to form the telomer 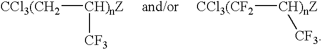
Products having n being at least 2 can be formed when utilizing an excess of the taxogen as compared to the telogen. For example, at least a 2:1 mole ratio of the taxogen to the telogen can be utilized to obtain products having n being at least 2. For example and by way of example only, at least two moles of the taxogen trifluoropropene can be exposed to at least one mole of the telogen (CF₃)₂CFl to form one or both of the telomers

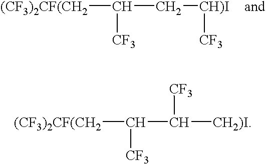

In additional embodiments initiator 6 may be provided to reactor 8 during the exposing of the reagents. Initiator 6 can include thermal, photochemical (UV), radical, and/or metal complexes, for example, including a peroxide such as di-tert-butyl peroxide. Initiator 6 can also include catalysts, such as Cu. Initiator 6 and telogen 4 can be provided to reactor 8 at a mole ratio of initiator 6 to taxogen 2 of from between about 0.001 to about 0.05 and/or from between about 0.01 to about 0.03, for example.

According to exemplary embodiments, various initiators 6 and telogens 4 can be used to telomerize taxogen 2 as referenced in Table 1 below. Telomerizations utilizing photochemical and/or metal-complex initiators 6 can be carried out in batch conditions using Carius tube reactors 8. Telomerizations utilizing thermal and/or peroxide initiators 6 can be carried out in 160 and/or 500 cm³ Hastelloy reactors 8. Telogen 4 (neat and/or as a peroxide solution) can be provided as a gas at a temperature from about 60° C. to about 180° C. and a telogen 4 [T]₀/taxogen 2 [Tx]₀ initial molar ratio R₀ can be varied from 0.25 to 1.5 and the reaction time from 4 to 24 hrs as dictated in Table 1 below. The product mixture can be analyzed by gas chromatography and/or the product can be distilled into different fractions and analyzed by ¹H and ¹⁹F NMR and/or ¹³C NMR. MonoAdduct (n=1) and DiAdduct (n=2) products can be recognized as shown in the Tables below.

TABLE 1
Telomerization of Trifluoropropene Taxogen

| | | | | | | | | | Yield (%) by GC^{c} | | |
|---|---|---|---|---|---|---|---|---|---|---|---|
| | | | | | | P (bars) | | % Conv. of | | MonoAdduct | DiAdduct |
| Run^{a} | Init.^{d} | R₀^{b} | C₀^{b} | T (° C.) | tᵣ (hrs) | max | min | Taxogen | Telogen | (n = 1) | (n = 2) |
| 1 | Therm | 0.50 | — | 160 | 20 | 22 | 17 | 79.2 | 27.6 | 51.9 | 20.5 |
| 2 | Therm | 0.25 | — | 160 | 20 | 39 | 34 | 36.8 | 52.8 | 26.2 | 21 |
| 3 | Therm | 0.50 | — | 180 | 22 | 30 | 11 | 73.4 | 2.4 | 65.9 | 31.2 |
| 4 | Perk | 0.50 | 0.03 | 62 | 20 | 7 | 5 | 79.2 | 23.8 | 35.4 | 40.8 |
| 5 | AIBN | 0.50 | 0.03 | 82 | 18 | 10 | 7 | 79.2 | 17.4 | 38.8 | 42 |
| 6 | TRIG | 0.50 | 0.03 | 134 | 6 | 16 | 0.6 | 89.6 | 3.7 | 19 | 63.8 |
| 7 | DTBP | 0.50 | 0.03 | 140 | 6 | 17 | 0.2 | 97.9 | 3.7 | 19 | 63.8 |
| 8 | DTBP | 0.50 | 0.03 | 143 | 4 | 19 | 0.8 | 94.3 | 9.6 | 21 | 66.6 |
| 9 | DTBP | 1.4 | 0.03 | 150 | 4 | 13 | 1.1 | 95.2 | 22.5 | 54.4 | 15.7 |
| 10 | DTBP | 0.75 | 0.03 | 145 | 4 | 20 | 3.0 | 93.8 | 6.8 | 34.1 | 49.0 |
| 11 | DTBP | 1.2 | 0.03 | 150 | 4 | 20 | 5.0 | 90.0 | 14.9 | 46.3 | 33.4 |
| 12 | DTBP | 1.4 | 0.03 | 150 | 4 | 21 | 3.5 | 95.0 | 12.6 | 54.1 | 28.6 |
| 13 | DTBP | 1.5 | 0.03 | 150 | 4 | 19 | 5.0 | 95.0 | 24.6 | 43.9 | 28.3 |

^{a}Telogen can be C₆F₁₃I in Runs Nos 1-9 and (CF₃)₂CFI in Runs No 10-13
^{b}R₀ = [T]₀/[Tx]₀; C₀ = [In]₀/[Tx]
^{c}Heavy TFP telomers (n > 2) can make up remainder of product
^{d}Initiators can be Perk. 16s(t-butyl cyclohexyl dicarbonate); AIBN; Trig. 101 (2,5-bis-(t-butyl peroxy)-2,5-dimethylhexane); and DTBP

TABLE 2
Telomerization of Pentafluoropropene Taxogen^{f}

| | | | | | | | Yield (%) by GC^{j} | | |
|---|---|---|---|---|---|---|---|---|---|
| | | | | | | % Conv. of | | MonoAdduct | DiAdduct |
| Run^{g} | Init.^{h} | R₀ⁱ | C₀ⁱ | T (° C.) | tᵣ (hrs) | Taxogen | Telogen | (n = 1) | (n = 2) |
| 1 | DTBP | 1.4 | 0.03 | 143 | 4 | <8 | 62.5 | 7.9 | 6.1 |
| 2 | DTBP | 1.4 | 0.03 | 143 | 4 | <5 | 82.8 | 5.1 | 1.1 |
| 3 | TRIG. 101 | 1.4 | 0.03 | 150 | 4 | <5 | 85.9 | 6.4 | 3.8 |
| 4 | TRIG. A80 | 1.4 | 0.03 | 180 | 5 | <10 | 63.4 | 4.9 | 1.6 |
| 5 | TRIG. A80 | 1.4 | 0.05 | 200 | 72 | <15 | 44.8 | 6.1 | 3.7 |
| 6 | TRIG. A80 | 1.4 | 0.06 | 220 | 48 | — | 50.7 | 3.2 | 1.4 |
| 7 | TRIG. A80 | 1.0 | 0.07 | 220 | 48 | — | 60.4 | 1.2 | 4.5 |
| 8 | TRIG. A80 | 0.5 | 0.08 | 220 | 48 | — | 41.7 | 1.2 | 2.8 |
| 9 | DIAD | 1.4 | 0.06 | 220 | 48 | — | 42.8 | 0.9 | 2.5 |
| 10 | DIAD | 1.0 | 0.06 | 220 | 48 | — | 42.7 | 0.8 | 1.8 |
| 11 | DIAD | 0.5 | 0.06 | 220 | 48 | — | 45.2 | 0.7 | 1.5 |
| 12 | CuCl | 1.4 | 0.4 | 140 | 48 | — | 20.2 | 0.1 | 0.2 |
| 13 | FeCl₂/benz | 1.4 | 0.4 | 140 | 48 | — | 14.8 | — | — |
| 14 | (PH₃P)₄Pd | 1.4 | 0.4 | 140 | 48 | — | 15.3 | 0.1 | 0.4 |
| 15 | Fe(II)acetate | 1.4 | 0.4 | 140 | 48 | — | 56.6 | 0.1 | 0.1 |

^{f}Telomerization of PFP with Rfl telogens at different reaction conditions (Hastelloy 160 cc reactor for runs 1-5 and 8 cc Carius tube for runs 6-15)
^{g}Rf is C₆F₁₅ except for run 2 where it is C3F7.
^{h}DTBP-di = tert-butyl peroxide; TRIG. 101-2,5-bis(tert-butylperoxy)2,5-dimethylhexane; TRIG A80-tert-butyl hydroxyperoxide; DIAD - diisopropyl azodicoarboxylate
ⁱR₀ = [T]₀/[Tx]₀; C₀ = [In]₀/[Tx], j) The remaining part is I₂ and/or heavy PFP telomers.

TABLE 3
Telomerization of PFP with non-fluorinated telogens (XY)^{k}

| | | | | | Yield (% by GC)ⁿ | | | |
|---|---|---|---|---|---|---|---|---|
| Run^{l} | Telogen | R₀^{m} | C₀^{m} | t_{R} (hours) | XY | n = 1 | n = 1 | n = 3 |
| 1 | HP(O)(OEt)₂ | 1.4 | 0.07 | 48 | 34.8 | 16.2 | 8.6 | 3.3 |
| 2 | BrCF₂CHClBr | 1.4 | 0.03 | 48 | 22.7 | 1.8 | 0.8 | — |
| 3 | CBrCl₃ | 1.4 | 0.03 | 48 | 77.8 | 0.3 | 0.3 | — |
| 4 | CHCl₃ | 1.4 | 0.05 | 48 | 18.1 | 27.1 | 12.0 | 6.3 |
| 5 | HS(CH₂)₂OH | 1.4 | 0.05 | 15 | 15.5 | 23.9 | 13.4 | — |

^{k}initiator can be DTBP; solvent CH₃CN at 50% (wt./wt.); Temperature 143° C.;
^{l}runs 1-4 in 8 cc Carious tube, run 5 in Hatelloy reactor
^{m}R₀ = [T]₀/[Tx]₀; C₀ = [In]₀/[Tx]
ⁿfor run No. 5, (% wt by distillation): HSR-18.2; n = 1-50.1, n = 2-28.3

TABLE 4
Cotelomerization of PFP with VDF and TFP^{o}

| | | | | | Conv vs | | | | | | |
|---|---|---|---|---|---|---|---|---|---|---|---|
| | Feed (mol %) | | In cotelomer (mol %) | | SM % | Yield (% by GC) | | | Yield (% by distillation) | | |
| Run | PFP | coM₂ | PFP | coM₂ | (wt./wt.) | R_{f}I | n = 1 | n = 2 | R_{f}I | n = 1 | n = 2 |
| 1 | 85 | VDF-15 | <3 | 98 | 33.2 | 57.8 | 6.3 | 4.7 | 85.3 | 18.5 | 12.8 |
| 2 | 85 | TFP-15 | 39 | 61 | 51.9 | 45.9 | 24.2 | 3.0 | 55.1 | 32.9 | 6.8 |

^{o}Runs performed in 160 cc Hastelloy reactor with DTBP initiator (3 mol %); R_{f}I = C₆F₁₃I; R₀ = 1.0; T = 145° C.; T_{R} = 5 hours

1-17. (canceled)

18. A telomerization process comprising exposing at least one CF₃-comprising taxogen to a fluorine-comprising telogen to produce a telomer, wherein the fluorine-comprising telogen comprises at least two fluorine atoms.

19. The process of claim 18 wherein the CF₃-comprising taxogen is pentaluoropropene.

20. The process of claim 18 wherein the fluorine-comprising telogen is (CF₃)₂CFl.

21. The process of claim 18 wherein the exposing the CF₃-comprising taxogen to the fluorine-comprising telogen is in the presence of an initiator.

22. The process of claim 21 wherein the initiator comprises a peroxide.

23. The process of claim 22 wherein the peroxide comprises di-tert-butyl peroxide.

24. The process of claim 22 wherein the exposing occurs within a reactor and the initiator and telogen are provided to the reactor, a mole ratio of the initiator to the telogen being between about 0.001 and about 0.05.

25. The process of claim 24 wherein the mole ratio of the initiator to the telogen is between about 0.01 and about 0.03.

26. The process of claim 19 wherein the exposing occurs within a reactor, a temperature within the reactor during the exposing being from about 130° C. to about 150° C.

27. The process of claim 18 wherein:
the CF₃-comprising taxogen is pentafluoropropene; and
the telomer comprises 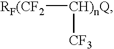
wherein:
the R_{F} group comprises at least two fluorine atoms;
n is at least 1; and
the Q group comprises one or more atoms of the periodic table of elements.

28. The process of claim 18 wherein:
the CF₃-comprising taxogen is pentafluoropropene;
the fluorine-comprising telogen is (CF₃)₂CFl; and
a mole ratio of the taxogen to the telogen is from about 2:1 to about 4:1.
